(19) United States
(12) Patent Application Publication
(10) Pub. No.: US 2012/0064125 A1
(43) Pub. Date: Mar. 15, 2012

(54) AGR-MEDIATED INHIBITION OF METHICILLIN RESISTANT STAPHYLOCOCCUS AUREUS

(76) Inventor: Alexander R. Horswill, Coralville, IA (US)

(21) Appl. No.: 13/257,548

(22) PCT Filed: Apr. 19, 2010
(86) PCT No.: PCT/US2010/031595

§ 371 Date: Dec. 1, 2011

Related U.S. Application Data
(60) Provisional application No. 61/170,349, filed on Apr. 17, 2009.

(51) Int. Cl. A61K 38/02; A61K 9/00; A01P 1/00; A61P 31/04; A01N 37/18

(52) U.S. Cl. 424/400; 514/2.7

(57) ABSTRACT

The present invention involves the use of activators of bacterial Agrquoroum-sensing systems to prevent or reverse biofilm formation in methicillin resistant Staphylococcus aureus (MRSA), or to restore sensitivity of MRSA bio films to antibiotics.

### BACKGROUND OF THE INVENTION

1. Field of the Invention

The invention generally concerns methods and compositions for the inhibition of biofilms. More specifically, the invention addresses the use of Agragonists to block or reverse methicillin resistant Staph. aureus biofilm formation and/or restore sensitivity to antibiotics.

2. Description of Related Art

Most bacteria have an inherent ability to form surface-attached communities of cells called biofilms (Davey and O'Toole, 2000). The opportunistic pathogen Staphylococcus aureus can form biofilms on many host tissues and implanted medical devices often causing chronic infections (Furukawa et al., 2006; Parsek and Singh, 2003; Harris and Richards, 2006; Costerton, 2005). The challenge presented by biofilm infections is the remarkable resistance to both host immune responses and available chemotherapies (Patel, 2005; Leid et al., 2002), and estimates suggest that as many as 80% of chronic bacterial infections are biofilm associated (Davies, 2003). In response to certain environmental cues, bacteria living in biofilms are capable of using active mechanisms to leave biofilms and return to the planktonic (free-living) state in which sensitivity to antimicrobials is regained (Fux et al., 2004; Boles et al., 2005; Hall-Stoodley and Stoodley, 2005). The emerging spread of MRSA strains poses an additional threat to public health (Lindsay, 2004; Vandenesch, 2003). USA300 and USA400 are the predominant MRSA strains circulating in North America, implicated in outbreaks of community-onset infections, leading to significant morbidity and mortality.

“Quorum-sensing” is a type of decision-making process used by decentralized groups to coordinate behavior. Many species of bacteria use quorum-sensing to coordinate their gene expression according to the local density of their population. Studies on the opportunistic pathogen Pseudomonas aeruginosa indicate that quorum-sensing is required to make a robust biofilm under some growth conditions (Davies et al., 2003). Surprisingly, the opposite is true in S. aureus, as the presence of an active quorum-sensing impedes attachment and development of a biofilm (Vuong et al., 2000; Beenken et al., 2003), with one study by Yarwood et al. (2004) showing that bacteria dispersing from biofilms displayed high levels of Agractivity, while cells in a biofilm had predominantly repressed Agrsystems.

The prevalence of methicillin-resistant Staphylococcus aureus (MRSA) infection has been growing steadily for more than a decade and in particular among those involved in orthopaedic trauma and joint replacement (Segawa et al., 1999; Zimmerli et al., 2004). These infections are associated with high morbidity and mortality, and they exert a tremendous financial burden on the healthcare system (Barberan, 2006). Treatment options require complex and expensive reconstructions and are further complicated by the antibiotic tolerance exhibited by bacterial growth on the foreign body. This resistance to treatment is primarily due to the development of a bacterial biofilm on the implant material (Zimmerli et al., 2004; Costerton, 2005). Biofilms are defined as a community of bacteria attached to a surface and encased in an exo-polymer matrix.

S. aureus pathogenicity and biofilm development is controlled by cell-to-cell communication, a ubiquitous regulatory mechanism that is often called quorum-sensing. During growth, S. aureus cells synthesize and secrete an autoinducing peptide (AIP) signal that accumulates in the extracellular environment. Once AIP reaches a critical concentration, the signal binds to a surface receptor and activates a regulatory cascade called the accessory gene regulator or Agrsystem, resulting in the increased expression of invasive factors, including toxins, hemolysins, proteases, and other tissue-degrading enzymes (Novick and Geisinger, 2008). To a lesser extent, the Agrsystem also decreases the expression of surface adhesins. Recently, the inventor reported that the activation of the Agrsystem in established biofilms triggers a dispersal pathway, detaching cells from a surface-bound biofilm and reverting them to a planktonic, antibiotic-susceptible state (Boles and Horswell, 2008).

Importantly, the type of S. aureus strains causing biofilm infections is evolving. There are increasing reports of methicillin-resistant S. aureus (MRSA) variants being isolated from infected prosthetic joints (Stoodley et al., 2008; Bassetti et al., 2005). This trend is concerning as MRSA infections result in worse patient outcomes and exert a higher economic burden than comparable methicillin-susceptible S. aureus (MSSA) infections (Lodise, Jr. and McKinnon, 2007). In addition, the identification of new MRSA strains in community settings, so called community-associated MRSA (CA-MRSA), has also altered the landscape of orthopaedic pathogens (Marcotte and Trzeciak, 2008). These new CA-MRSA isolates are remarkably invasive and cause more severe and devastating disease compared to traditional healthcare-associated MRSA (Klevens et al., 2007; Voyich et al., 2005). Of the CA-MRSA isolates, the pulse field gel group called “USA300” has emerged as the dominant isolate causing the majority of recently reported infections (Miller and Diep, 2008). Indeed, outbreaks of USA300 in prosthetic joint infections have also been reported (Kourbatova et al., 2005), and there are increasing reports of USA300 isolates in other biofilm infections (Hague et al., 2007; Seybold et al., 2007). Due to the enhanced ability of CA-MRSA isolates to cause post-operative orthopaedic complications, new treatment strategies are necessary to combat this emerging, aggressive pathogen.

### SUMMARY OF THE INVENTION

Thus, in accordance with the present invention, there is provided a method of inhibiting a methicillin resistant Staphylococcus aureus (MRSA) biofilm comprising contacting a biofilm-forming MRSA with an activator of an Agrquorum-sensing system. The Agrquorum-sensing system may be Agr-I, Agr-II, Agr-III or Agr-IV. The activator may be an autoinducing peptide (AIP). The method may further comprise contacting said MRSA with an antibiotic or antiseptic agent Inhibiting may comprise inhibiting MRSA biofilm formation, inhibiting MRSA biofilm growth, reducing MRSA biofilm size or promoting detachment of MRSA from a formed biofilm.

The MRSA biofilm or biofilm-forming MRSA may be located in a subject, such as a mammalian subject, including a human subject. The subject may comprises an in-dwelling medical device, such as an implant, a catheter, a pump, endotracheal tube, a nephrostomy tube, a stent, an orthopedic device, a suture, a or prosthetic valve. The catheter may be a vascular catheter, an urinary catheter, a peritoneal catheter, an epidural catheter, a central nervous system catheter, central venous catheter, an arterial line catheter, a pulmonary artery catheter, or a peripheral venous catheter. The method may thus further comprise coating the in-dwelling medical device with said inhibitor prior to implantation. The MRSA biofilm or biofilm-forming MRSA may be located on a wound dressing, or on a tissue surface, such as a heart valve, bone or epithelia.

Alternatively, the MRSA biofilm or biofilm-forming MRSA may be located on an inanimate surface, such as a floor, a table-top, a counter-top, a medical device surface, a wheelchair surface, a bed surface, a sink, a toilet, a filter, a valve, a coupling, or a tank. The biofilm may also be located in an industrial system, such as a heating/cooling system, a water provision or purification system, or a medical pump system.

In another embodiment, there is provided a method of preventing a methicillin resistant Staphylococcus aureus (MRSA) biofilm formation secondary to nosocomial infection in a subject comprising administering to said subject an activator of an Agrquorum-sensing system in combination with an antibiotic. The nosocomial infection may be pneumonia, bacteremia, a urinary tract infection, a catheter-exit site infection, and a surgical wound infection.

In still another embodiment, there is provided a method of restoring antibiotic sensitivity to a methicillin resistant Staphylococcus aureus (MRSA) bacterium located in a biofilm comprising contacting said MRSA with an activator of an Agrquorum-sensing system. The method may further comprise administerting an antibiotic to said subject.

As used herein the specification, “a” or “an” may mean one or more. As used herein in the claim(s), when used in conjunction with the word “comprising,” the words “a” or “an” may mean one or more than one. The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIGS. 1A-B. USA300 biofilm dispersal from a glass surface. USA300 strain LAC with plasmid pALC2084 was inoculated into a once-through flow cell with a glass substratum and incubated for two days. AIP-I (˜50 nM) was added to the flow cell media and biofilm integrity was monitored for 20 hr. For each image depiction, a z series of images was obtained with CLSM and reconstructed with Volocity software, and each side of a grid square is 20 μm in the image reconstruction. (FIG. 1A) Wild-type LAC with pALC2084. (FIG. 1B) LAC ΔAgr:Tet mutant with pALC2084.

FIG. 2. Flow cell for testing alternative abiotic surfaces. A BioSurface Technologies FC270 flow cell chamber was adapted for testing titanium as a USA300 biofilm surface. Pure titanium disks were machined to a 10 mm×2 mm dimension to fit in the flow chamber. Standard polycarbonate disks are also shown.

FIGS. 3A-C. Comparison of USA300 biofilm maturation on different surface chemistries. LAC was grown in flow chambers with either glass, polycarbonate, or titanium as a substratum for five days. CLSM images were obtained at both two and five days, and COMSTAT analysis was performed. (FIG. 3A) Average biofilm thickness and maximum thickness in μm. (FIG. 3B) Total biomass in μm³/μm². (FIG. 3C) Amount of surface coverage of the biofilm layer in contact with either glass, polycarbonate, or titanium. Error bars represent standard deviation.

FIGS. 4A-B. Protease and DNaseI treatment of USA300 titanium biofilms. Biofilms of LAC with plasmid pCM12 were grown for two days on titanium. Biofilms were either untreated or grown in the presence of Proteinase K or DNaseI. For treatments, Proteinase K was added to 2 μg/mL and DNaseI was added to 0.5 Units/mL to the flow cell media. At 6 and 22 hr, CLSM images were obtained and analyzed. (FIG. 4A) Representative CLSM image reconstructions generated from a z series. Each side of the image grid square is 20 μm. (FIG. 4B) COMSTAT analysis of images represented in terms of average thickness (μm) and biomass (μm³/μm²). Green triangles represent untreated biofilm, red circles represent DNaseI treatment, and black squares represent Proteinase K treatment. Error bars represent standard deviation.

FIG. 5. Antibiotic susceptibility of USA300 biofilms detached from titanium. Biofilm bacteria (grey circles) were grown in flow cells containing removable titanium coupons. After three days growth, the coupons were removed, biofilms (grey circles) were exposed to increasing concentrations of rifampicin/levofloxacin at indicated concentrations (in μg/mL), and surviving CFU's were determined. In parallel, biomass was detached (white triangles, dashed-line error bars) from the coupons with AIP-I addition and collected from flow cell effluents. The detached biomass was treated with identical rifampicin/levofloxacin concentrations and surviving CFU's determined. As a control, planktonic bacteria (black diamonds) were also tested for rifampicin/levofloxacin resistance under the same conditions. Graph shows the mean of three experiments and error bars show standard deviation.

FIGS. 6A-B. USA300 biofilm dispersal from a titanium surface. Biofilms of LAC with plasmid pCM12 were grown for two days in the modified flow cell chamber (FIG. 2) that contains titanium as a substratum. AIP-I was added to the flow cell media at ˜50 nM final concentration, and biofilm integrity was monitored for another 20 hr. Each image depiction is a reconstruction of a z series of CLSM images, and each side of a grid square is 20 μm. (FIG. 6A) Wild-type LAC with pCM12. (FIG. 6B) LAC ΔAgr::Tet mutant with pCM12.

### DETAILED DESCRIPTION OF THE INVENTION

The majority of studies on biofilm detachment have focused on factors capable of initiating the process, such as nutrient availability (Hunt et al., 2004; Sauer et al., 2004), nitric oxide exposure (Barraud et al., 2006), oxygen tension (Thormann et al., 2005), iron salts (Musk et al., 2005), chelators (Banin et al., 2006), and signaling molecules (Morgan et al., 2006; Rice et al., 2005; Dow et al., 2003; Thormann et al., 2006). Alternatively, detachment studies have addressed effector gene products that contribute to the dissolution of the biofilm, including surfactants (Boles et al., 2005; Vuong et al., 2000; Irie et al., 2005; Davey et al., 2003), hydrolases (Kaplan et al., 2004; Kaplan et al., 2003), proteases (Chaignon et al., 2007; O'Neill et al., 2007; Rohde et al., 2007), and DNase (Whitchurch et al., 2002).

S. aureus has been reported to form biofilms through an ica-dependent mechanism suggesting that PIA could have a role in detachment (O'Gara, 2007; Cramton et al., 1999). The inventor has observed no defect in microtiter or flow cell biofilm formation using an ica mutant, which supports the growing evidence that PIA is not a major matrix component of S. aureus biofilms, as exogenous addition of dispersin B, an N-acetyl-glucosaminidase capable of degrading PIA, has little effect on established biofilms of SH1000 and other S. aureus strains (Izano et al., 2008). In contrast, dispersin B does detach S. epidermidis biofilms indicating a more significant role for PIA in the S. epidermidis matrix structure (Izano et al., 2008). The inventor's experiments with proteinase K and the S. aureus proteases indicate that some proteinaceous material is important for SH1000 biofilm integrity, and this result supports a number of recent studies demonstrating that proteases can inhibit biofilm formation or detach established biofilms from many S. aureus strains ((Toledo-Arana et al., 2005; Chaignon et al., 2007; O'Neill et al., 2007; Rohde et al., 2007). It is not clear whether Agr-mediated detachment will function in S. aureus strains that produce an ica-dependent biofilm.

There is increasing interest in understanding how bacteria detach from biofilms and initiate colonization of new surfaces. The regulation of quorum-sensing systems may be one mechanism by which many bacteria control biofilm formation and dispersal. Quorum-sensing has been implicated in dispersal of biofilms formed by Yersinia pseudotuberculosis (Atkinson et al., 1999), Rhodobacter sphaeroides (Puskas et al., 1997), Pseudomonas aureofaciens (Zhang and Pierson, 2001), Xanthomonas capmestris (Dow et al., 2003), and Serratia marceascens (Rice et al., 2005). However, homoserine lactone signals play a divergent role in Pseudomonas aeuruginosa (Davies et al., 1998), Pseudomonas fluorescens (Allison et al., 1998), and Burkholderia cepacia (Huber et al., 2001), where the active versions of these quorum-sensing system are necessary for biofilm formation and robustness under some growth conditions. In both cases, it appears quorum-sensing plays a significant role in biofilm development and determining the environmental stimuli that modulate quorum-sensing activity will provide insight on bacterial colonization, detachment, and dispersal to new sites.

The inventor has previously demonstrated that activation of the Agrsystem in established biofilms is necessary for detachment (Boles & Horswill, 2008). This activation could be accomplished with exogenous AIP addition or by changing nutrient availability to the biofilm. The inventor also has demonstrated that Agr-mediated detachment requires the activity of extracellular proteases. Together, these findings suggest that Agrquorum-sensing is an important regulatory switch between planktonic and biofilm lifestyles that may contribute to bacterial dispersal and colonization of new sites. However, the inventor's prior work did not address whether methicillin resistant Staphylococcus aureus (MRSA) biofilms would similarly be inhibited by an activator of an Agrquorum-sensing system. As shown herein, despite the altered physiology of MRSA organisms, and their notorious resistance to even the most aggressive antibiotic treatments, MRSA biofilms are in fact inhibited by activators of Agrquorum-sensing systems.

### I. AgrQUORUM-SENSING SYSTEMS

A. Quorum-Sensing

Quorum-sensing is a type of decision-making process used by decentralized groups to coordinate behavior. Many species of bacteria use quorum-sensing to coordinate their gene expression according to the local density of their population. Similarly, some social insects use quorum sensing to make collective decisions about where to nest. In addition to its function in biological systems, quorum sensing has several useful applications for computing and robotics. Quorum sensing can function as a decision-making process in any decentralized system, as long as individual components have (a) a means of assessing the number of other components they interact with and (b) a standard response once a threshold number of components is detected.

Some of the best-known examples of quorum-sensing come from studies of bacteria. Bacteria use quorum-sensing to coordinate certain behaviors based on the local density of the bacterial population. Quorum-sensing can occur within a single bacterial species as well as between diverse species, and can regulate a host of different processes, essentially serving as a simple communication network. A variety of different molecules can be used as signals.

Three-dimensional structures of proteins involved in quorum-sensing were first published in 2001, when the crystal structures of three LuxS orthologs were determined by X-ray crystallography. In 2002, the crystal structure of the receptor LuxP of Vibrio harveyi with its inducer AI-2 (which is one of the few biomolecules containing boron) bound to it was also determined. AI-2 signalling is conserved among many bacterial species, including E. coli, an enteric bacterium and model organism for Gram-negative bacteria. Although this conservation has suggested that autoinducer-2 could be used for widespread interspecies communication, a comparative genomic and phylogenetic analysis of 138 genomes of bacteria, archaea, and eukaryotes did not support the concept of a multispecies signaling system relying on AI-2 outside Vibrio species.

The S. aureus quorum-sensing system is encoded by the accessory gene regulator (Agr) locus and the communication molecule that it produces and senses is called an autoinducing peptide (AIP), which is an eight-residue peptide with the last five residues constrained in a cyclic thiolactone ring (Ji et al., 1997) mechanism that requires multiple peptidases (Kavanaugh et al., 2007; Qiu et al., 2005). Once AIP reaches a critical concentration, it binds to a surface histidine kinase receptor, initiating a regulatory cascade that controls expression of a myriad of virulence factors, such as proteases, hemolysins, and toxins (Novick, 2003). A recent study by Yarwood et al. (2004) raised the possibility that the Agrquorum-sensing system is involved in biofilm detachment. That study demonstrated that bacteria dispersing from biofilms displayed high levels of Agractivity, while cells in a biofilm had predominantly repressed Agrsystems. These findings correlate well with prior data indicating that Agr-deficient S. aureus strains form more robust biofilms compared to wild-type strains (Vuong et al., 2000; Beenken et al., 2003). However, the effects of Agrmodulation of biofilm formation and maintenance have yet to be explored.

Bacteria that use quorum sensing constantly produce and secrete certain signaling molecules (called autoinducers or pheromones). These bacteria also have a receptor that can specifically detect the signaling molecule (inducer). When the inducer binds the receptor, it activates transcription of certain genes, including those for inducer synthesis. There is a low likelihood of a bacterium detecting its own secreted AHL. Thus, in order for gene transcription to be activated, the cell must encounter signaling molecules secreted by other cells in its environment. When only a few other bacteria of the same kind are in the vicinity, diffusion reduces the concentration of the inducer in the surrounding medium to almost zero, so the bacteria produce little inducer. However, as the population grows the concentration of the inducer passes a threshold, causing more inducer to be synthesized. This forms a positive feedback loop, and the receptor becomes fully activated. Activation of the receptor induces the up regulation of other specific genes, causing all of the cells to begin transcription at approximately the same time.

B. Bacteria

Staphylococcus aureus is a major human pathogen, causing a wide variety of illnesses ranging from mild skin and soft tissue infections and food poisoning to life-threatening illnesses such as deep post-surgical infections, septicaemia, endocarditis, necrotizing pneumonia, and toxic shock syndrome. These organisms have a remarkable ability to accumulate additional antibiotic resistance determinants, resulting in the formation of multiply-drug-resistant strains. Methicillin, being the first semi-synthetic penicillin to be developed, was introduced in 1959 to overcome the problem of penicillin-resistant S. aureus due to β-lactamase (penicillinase) production (Livermore, 2000). However, methicillin-resistant S. aureus (MRSA) strains were identified soon after the introduction of methicillin (Barber, 1961; Jevons, 1961). MRSA have acquired and integrated into their genome a 21- to 67-kb mobile genetic element, termed the Staphylococcus cassette chromosome mec (SCCmec) that harbors the methicillin resistance (mecA) gene and other antibiotic resistance determinants (Ito et al., 2001; Ito et al., 2004; Ma et al., 2002). The mecA gene encodes an altered additional low affinity penicillin-binding protein (PBP2a) that confers broad resistance to all penicillin-related compounds including cephalosporins and carbapenems that are currently some of the most potent broad-spectrum drugs available (Hackbarth & Chambers, 1989). Since their first identification, strains of MRSA have spread and become established as major nosocomial (hospital-acquired (HA)-MRSA) pathogens worldwide (Ayliffe, 1997; Crossley et al., 1979; Panlilio et al., 1992; Voss et al., 1994). Recently, these organisms have evolved and emerged as a major cause of community-acquired infections (CA-MRSA) in healthy individuals lacking traditional risk factors for infection, and are causing community-outbreaks, which pose a significant threat to public health (Begier et al., 2004; Beilman et al., 2005; Conly et al., 2005; Gilbert et al., 2006; Gilbert et al., 2005; Harbarth et al., 2005; Holmes et al., 2005; Issartel et al., 2005; Ma et al., 2005; Mulvey et al., 2005; Robert et al., 2005; Said-Salim et al., 2005; Vandenesch et al., 2003; Vourli et al., 2005; Wannet et al., 2005; Wannet et al., 2004; Witte et al., 2005; Wylie & Nowicki, 2005).

The incidence of MRSA infection has greatly increased over the past 5 years due to the spread of community-associated MRSA. The two predominant strains of CA-MRSA circulating in North America belong to pulsed-field gel types USA300 and USA400 strains according to the CDC classification. The USA300 and USA400 stains have been associated with serious infections including soft tissue abscesses, cellulitis, necrotizing fasciitis, severe multifocal osteomyelitis, bacteremia with Waterhouse-Frederickson syndrome, septic shock and necrotizing pneumonia (Beilman et al., 2005; CDC, 2003; Conly et al., 2005; Francis et al., 2005; Kazakova et al., 2005). Of greater concern is the high transmissibility of USA300 and the link between both USA300 and USA400 and disease outbreaks worldwide (Kazakova et al., 2005; Pan et al., 2003; Tenover et al., 2006). Another alarming observation is that community-associated MRSA strains, in particular USA300, are being reported as causing hospital acquired MRSA infections as well (Bratu et al., 2005; Chalumeau et al., 2005; Linde et al., 2005; Naas et al., 2005).

The USA400 strain is represented by strain MW2, isolated in 1998 in North Dakota from a pediatric patient with fatal septicaemia (1999). The MW2 genome has been fully sequenced and shown to contain 4 genomic islands (ν Sa3, ν Sa4, ν Saα and ν Saβ) 2 prophages (φSa2mw and φSa3mw) and an SCCmec element (IVa), all of which contribute to its virulence (Baba et al., 2002). MW2 is a hypervirulent strain carrying a large number of toxin genes, including new allelic forms of enterotoxins L (sel2) and C (sec4) on ν Sa3, 11 putative exotoxins (set16-26) on ν Saα, lukD and lukE leukotoxins on νSaβ, enterotoxin A (sea), Q (seq) and 2 new allelic forms of enterotoxin G (seg2) and K (sek2) on prophage φSa3mw (Baba et al., 2002). Prophage φSa2mw harbours the lukS-PV and the lukF-PV genes, encoding the PVL components (Baba et al., 2002). Also found in the MW2 genome, but not associated with genomic islands or prophages, are the genes encoding γ-hemolysin (hlg) and enterotoxin H (seh) (Baba et al., 2002).

The USA300 strain, represented by strain FPR3757, was isolated in 2000 from an inmate in a California prison (2001). It has been sequenced and similar to USA400, found to contain multiple genetic elements which contribute to virulence, including an SCCmec element (IVa), 2 prophages (φSa2usa and φSa3usa), 3 pathogenicity islands (SaPI5, ν Saα and ν Saβ) and the Arginine Catabolic Mobile Element (ACME) (Diep et al., 2006). In contrast to the USA400 genome, which bears a large number of toxin genes, the genome of USA300 carries a smaller number of toxin genes, including enterotoxins K and Q on SaPI5 and set30-39 on ν Saα. Prophage φSa2usa is very similar in structure to φSa2mw and, likewise, carries the PVL genes, lukS-PV and lukF-PV. Unique to the USA300 genome is the presence of a 30.9 kb ACME complex. The ACME complex is integrated into the chromosome at the same attachment site as SCCmec and contains an arc gene cluster, encoding an arginine deiminase pathway, as well as a putative oligopeptide permease operon, Opp (Diep et al., 2006). In addition to USA300 strain, the ACME complex has been found in Staphylococcus capitis and Staphylococcus epidermidis, but due to its high frequency of occurrence in S. epidermidis it is believed to have transferred to USA300 from this species (Diep et al., 2006).

The USA300 and USA400 strains belong to multi-locus sequence typing (MLST) type 8 (ST8) and ST1, respectively and both carry Panton-Valentine leukocidin (PVL) genes and SCCmec type IVa. To date, there is no rapid way to identify and characterize CA-MRSA, but rather numerous time and labor intensive molecular characterization tests. An accurate and rapid PCR based assay, able to distinguish USA300 and USA400 isolates from other MRSA, would facilitate in the identification of outbreaks, treatment of patients and aid in the implementation of control measures designed to limit the spread of these serious pathogens.

C. Activators of Quorum Sensing Systems

i. AIP Compositions

In certain embodiments, the present invention concerns compositions comprising so-called “autoinducing peptides” that are involved in quorum-sensing in bacteria. An interesting feature of the S. aureus Agrsystem is the variation among strains (Novick, 2003). There are four different classes of Agrsystems each recognizing a unique AIP structure (referred to as Agr-I, Agr-II, Agr-III, and Agr-IV; similarly, their cognate signals are termed AIP-I through AIP-IV). Through a fascinating mechanism of chemical communication, these different AIP signals cross-inhibit the activity of the others with surprising potency, presumably giving a competitive advantage to the producing S. aureus strain. Indeed, Agrinterference has been observed with in vivo competition experiments (Fleming et al., 2006), and the addition of an inhibitory AIP will block development of an acute infection (Wright et al., 2005).

Among the four AIP classes, the five-residue thiolactone ring structure is always conserved, while the other ring and tail residues differ (Malone et al., 2007). Similarly, the proteins involved in signal biosynthesis and surface receptor binding also show variability (Wright et al., 2004; Zhang and Ji, 2004). In Agrinterference, there are three classes of cross-inhibitory groups: AIP-I/IV, AIP-II, and AIP-III. Since AIP-I and AIP-IV differ by only one amino acid and function interchangeably (Jarraud et al., 2000), they are grouped together. The three AIP groups all cross-inhibit each other with binding constants in the low nanomolar range (Lyon et al., 2002; Mayville et al., 1999). Interestingly, the typing of the four Agrsystems roughly correlates with specific classes of diseases (Jarraud et al., 2000; Jarraud et al., 2002), although the significance of this observation is unclear.

Studies that have relied on extracellular addition of AIPs have required chemical synthesis of the signal (Sung et al., 2006; Wright et al., 2005). While the strategy has been effective, it is prohibitive for many laboratories, impeding research on the AIP molecules. The AIPs can be purified from culture supernatants (Ji et al., 1997), but the yields are low and the procedures are labor-intensive, making this approach unattractive. The inventor also has reported on a convenient, enzymatic approach to generating AIP molecules (Malone et al., 2007) employing an engineered DnaB mini-intein from Synechocystis sp. strain PCC6803. The sequences of AIP-I to -IV are shown below:

| | | |
|---|---|---|
| AIP-I | YSTCDFIM | SEQ ID NO: 1 |
| AIP-II | GVNACSSLF | SEQ ID NO: 2 |
| AIP-III | INCDFLL | SEQ ID NO: 3 |
| AIP-IV | YSTCYFIM | SEQ ID NO: 4 |

For each peptide, a thiolactone bridge is formed between the C-terminal residues and the underlined internal cysteine reside. Methods of making such peptides are disclosed in PCT US2007/087663, incorporated herein by reference. Other related compounds are described in U.S. Pat. Nos. 6,953,833 and 6,337,385, and U.S. Patent Publication 2007/0185016, incorporated herein by reference.

In certain embodiments the AIP composition is provided in a biocompatible form. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In particular embodiments, biocompatible protein, polypeptide or peptide containing compositions will generally be proteins or peptides or synthetic proteins or peptides each essentially free from toxins, pathogens and harmful immunogens.

In certain embodiments and as described supra, AIP's may be purified. Generally, “purified” will refer to a protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

ii. Other Activators

Another class of activators for the present invention include inhibitors of the SigB system. One of the most important global regulators in S. aureus, the SigB system is an environmental sensing mechanism used by diverse Gram-positive bacteria to coordinate gene expression. Essentially SigB inhibition leads to Agrquorum-sensing activation—the two pathways have an inverse correlation. As such, SigB inhibition works in the same way as adding AIP. It activates the quorum-sensing system and disperses the biofilm. MRSA biofilms inhibited by SigB using a small molecule may result in the biofilm being completely dispersed.

The best characterized of these regulatory networks is from Bacillus subtilis and contains numerous proteins involved in sensing a variety of stresses, including heat, high salt, and alkaline shock conditions (Pane-Farre et al., 2006), and these signals are transmitted them through a cascade to activate SigB. Based on the presence of a SigB gene in other Gram positives, it has long been assumed that the system function is conserved throughout the Gram positives.

However, recent studies in S. aureus demonstrate that the activation mechanism and output regulon shares little resemblance to B. subtilis paradigm (Pane-Farre et al., 2006). In S. aureus, SigB regulates many factors related to virulence, such as carotenoid, hemolysins, extracellular invasive enzymes, polysaccharide intracellular adhesin (PIA), and biofilm formation (Kullik et al., 1997; Horsburgh et al., 2002; Rachid et al., 2000; Bischoff et al., 2004; Ziebandt et al., 2004; 2001; Gertz et al., 1999; Cheung et al., 1999). While many features of the B. subtilis and S. aureus Sigma B systems are different, the Rsb and SigB proteins are similar based on sequence identity. Based on the B. subtilis model, it is assumed that the S. aureus Rsb proteins operate as a protein-protein interaction cascade to modulate SigB activity. Briefly, under environmental stress conditions (heat, base, salt), RsbU dephosphorylates RsbV protein, allowing RsbV and RsbW to interact. With RsbW bound, SigB is free to activate transcription. Under normal growth conditions, RsbV remains phosphorylated, and RsbW functions as an anti-sigma factor and sequesters SigB. While genetic and molecular analysis supports this model, there is little biochemical evidence to verify it. Further, it is not clear how signals are transmitted into the RsbU protein. B. subtilis has a complex sensory component that is completely missing in S. aureus (Pane-Farre et al., 2006). Considering all the S. aureus virulence factors regulated by SigB, it is surprising that these basic features of the system remain unknown.

The role of SigB in S. aureus biofilm formation has also been controversial. Initial reports on S. aureus SigB defective strains indicated they were unable to form a biofilm (Rachid et al., 2000). However, a later study contradicted these reports and claimed the SigB biofilm phenotype was due to regulation of SarA (Valle et al., 2003), which is known to contain at least one SigB-dependent promoter. In S. epidermidis, it is known that SigB is required to express PIA (Knobloch et al., 2001; 2004), explaining the biofilm defect of SigB mutants in this organism. There has been speculation that SigB regulation of PIA also explains the S. aureus biofilm phenotypes, but growing number of clinical strains produce PIA-independent (ica-independent) biofilms (Izano et al., 2008; O'Neill et al., 2007), especially among the MRSA isolates. Interestingly, overexpression of SigB greatly improves attachment to various human matrices (Entenza et al., 2005). In the inventor's screens for biofilm defective S. aureus mutants, they found multiple insertions in the rsbUVW-sigB locus, and follow-up studies indicate that SigB is important for biofilm formation. Under certain conditions, such as SigB inactivation, high level production of extracellular enzymes ensues and biofilm formation is blocked, and thus the inventor speculates these enhanced exoenzyme levels are the reason for the biofilm phenotypes. Based on these observations, the inventor proposes a model to explain the role of SigB in biofilms. In brief, when an environmental cue induces the SigB system, S. aureus will preferentially form a biofilm, and when SigB is repressed, cells will remain planktonic or leave an established biofilm.

Thus, the present invention contemplates the use of inhibitors of the SigB pathway as a means for activating quorum-sensing in bacteria to prevent biofilms. Such inhibitors may be pharmaceutical “small molecules,” or them may be biologicals, as discussed below.

Antisense Constructs. An alternative approach to inhibiting SigB is antisense. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Ribozymes. Another general class of inhibitors is ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

RNAi. RNA interference (also referred to as “RNA-mediated interference” or RNAi) is another mechanism by the SigB system can be inhibited. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically-synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Treatment regimens would vary depending on the clinical situation. However, long term maintenance would appear to be appropriate in most circumstances. It also may be desirable treat hypertrophy with inhibitors of TRP channels intermittently, such as within brief window during disease progression.

Antibodies. In certain aspects of the invention, antibodies may find use as inhibitors. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference. “Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

### II. SCREENING METHODS

The present invention further comprises methods for identifying agents that inhibit the Agrquorum-sensing systems of MRSA. These assays may comprise random screening of large libraries of candidate substances; alternatively, the assays may be used to focus on particular classes or sequences of compounds selected with an eye towards structural attributes that are believed to make them more likely result in a particular biological function, such as antibiotic activity. One example would be mimetics of AIPs, while another would be a SigB-family inhibitor.

To identify a biologically active candidate substance, one generally will determine the a specific biological activity (e.g., cell or biofilm growth, biofilm formation, biofilm detachment) in the presence and absence of the candidate substance. For example, a method generally comprises:
(a) providing a candidate substance;
(b) admixing the candidate polypeptide with a biofilm-forming MRSA cell or MRSA biofilm, either in vitro or in a suitable experimental animal;
(c) measuring one or more quorum-sensing characteristics of the MRSA cell, MRSA biofilm or animal in step (b); and
(d) comparing the characteristic measured in step (c) with the characteristic of the MRSA cell, MRSA biofilm or animal in the absence of said candidate polypeptide, wherein a difference between the measured characteristics indicates that said candidate modulator is, indeed, a modulator of MRSA cell, MRSA biofilm or animal.
   It will, of course, be understood that such screening methods are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit or enhance Agrquorum sensing. It may prove to be the case that the most useful pharmacological compounds will be compounds that are structurally related to AIP peptides, such as those from S. aureus. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs that are more active or stable than the natural molecules, which have different susceptibility to alteration or which may affect the function of various other molecules. In one approach, one would generate a three-dimensional structure for a target molecule, or a fragment thereof.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Candidate substances may include fragments or parts of naturally-occurring compounds, or may be found as active combinations of known compounds, which are otherwise inactive. It is proposed that amino acid sequences isolated from natural sources, such as animals, bacteria, fungi, plant sources, may be assayed as candidates for the presence of potentially useful pharmaceutical agents. It will be understood that the pharmaceutical agents to be screened could also be derived or synthesized from chemical compositions or man-made compounds.

In addition to the modulating compounds initially identified, the inventor also contemplates that other sterically similar compounds may be formulated to mimic the key portions of the structure of the modulators.

An MRSA biofilm inhibitor according to the present invention may be one which exerts its activating effect upstream, downstream or directly on a a quorum sensing system. Regardless of the type of activator identified by the present screening methods, the effect of the activator by such a compound results in discernable biological changes compared to that observed in the absence of the added candidate substance.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates (e.g., multiwell plates), dishes and other surfaces such as dipsticks or beads.

One example of a cell free assay is a binding assay. While not directly addressing function, the ability of molecule to bind to a target in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

Methods may also involve the combination of a traditional antibiotic with an agent that inhibits or enhances Agrquorum sensing.

C. In Cyto Assays

The present invention also contemplates the screening of candidate substances for their ability to modulate quorum sensing pathways in MRSA cells. Various MRSA strains can be utilized for such screening assays, or bacterial cells specifically engineered for this purpose. For example, in some aspects, the effect of the candidate substances on MRSA cell or MRSA biofilm growth may be assessed. In still other cases cells for an in cyto assay may comprise a reporter gene indicating the activity or inhibition of a quorum sensing pathway. For instance, cells may be bacterial cells that express a reporter gene under the control of a promoter that responds to quorum sensing pathways. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In Vivo Assays

In vivo assays involve the use of various animal models. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound.

Treatment of these animals with candidate substances will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a candidate substance in vivo may involve a variety of different criteria. Also, measuring toxicity and dose response can be performed in animals in a more meaningful fashion than in in vitro or in cyto assays.

### III. METHODS

A. Methods of Treating Subjects

The present invention contemplates, in one embodiment, the treatment of subjects suffering from MRSA biofilm formation or at risk of biofilm formation due to various medical or environmental conditions. A variety of medical situations lend themselves to risk of MRSA biofilm involvement. For example, patients on chronic antibiotic therapy, immunosuppressed patients, patients having had surgery, and patients with traumatic wounds all are at risk of developing MRSA biofilm-type infections.

Administration of pharmaceutical compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions. Upon formulation, solutions will be administered in a manner compatible with the dosage formulation and in such amount as is therapeutically effective.

B. Pharmaceutical Formulations

Where clinical applications are contemplated, it will be necessary to prepare pharmaceutical compositions—AIPs and other Agrquorum-sensing signaling agents—in a form appropriate for the intended application. Generally, this will entail preparing compositions that are essentially free of pyrogens, as well as other impurities that could be harmful to humans or animals.

One will generally desire to employ appropriate salts and buffers to agents stable and allow for uptake by target cells. Aqueous compositions of the present invention comprise an effective amount of the agent to cells or a subject, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the agents of the present invention, its use in therapeutic compositions is contemplated. Supplementary active ingredients also can be incorporated into the compositions.

C. Combination Therapy

Antibiotic resistance represents a major problem in microbiology, and in particular, in the treatment of MRSA biofilms. A major goal of current research is to find ways to improve the efficacy of standard antibiotics, and one way is by combining such traditional therapies with a sensitizing or augmenting agent. Thus, in accordance with the present invention, one may kill bacteria, inhibit MRSA or MRSA biofilm growth, inhibit MRSA biofilm development or spread, induce detachment of a MRSA biofilm-involved bacterium or re-establish antibiotic sensitivity of a MRSA bacteria or MRSA biofilm, one would generally contact a “target” bacterium, biofilm or subject with an Agrquorum-sensing agent and at least one other agent. These compositions would be provided in a combined amount effective to achieve any of the foregoing goals. This process may involve contacting the MRSA bacteria, MRSA biofilm or subject with the Agrquorum-sensing agent and the other agent(s) or factor(s) at the same time. This may be achieved by contacting the MRSA with a single composition or pharmacological formulation that includes both agents, or by contacting the cell with two distinct compositions or formulations, at the same time, wherein one composition includes the Agrquorum-sensing agent and the other includes the other agent. Alternatively, the Agrquorum-sensing agent therapy treatment may precede or follow the other agent treatment by intervals ranging from minutes to weeks. In embodiments where the other agent and Agrquorum-sensing agent are applied separately to the MRSA bacteria, biofilm or subject, one would generally ensure that a significant period of time did not expire between the time of each delivery, such that the other agent and Agrquorum-sensing agent would still be able to exert an advantageously combined effect on the MRSA bacteria, MRSA biofilm or subject. In such instances, it is contemplated that one would contact both modalities within about 12-24 hours of each other and, within about 6-12 hours of each other, within about 6 hours of each other, within about 3 hours of each other or within about 1 hour of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of Agrquorum-sensing agent or the other agent will be desired. Various combinations may be employed, where Agrquorum-sensing agent is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. Antibiotics thay may be employed are include the aminoglycosides (Amikacin (IV), Gentamycin (IV), Kanamycin, Neomycin, Netilmicin, Paromomycin, Streptomycin (IM), Tobramycin (IV)), the carbapenems (Ertapenem (IV/IM), Imipenem (IV), Meropenem (IV)), Chloramphenicol (IV/PO), the fluoroquinolones (Ciprofloxacin (IV/PO), Gatifloxacin (IV/PO), Gemifloxacin (PO), Grepafloxacin* (PO), Levofloxacin (IV/PO), Lomefloxacin, Moxifloxacin (IV/PO), Norfloxacin, Ofloxacin (IV/PO), Sparfloxacin (PO), Trovafloxacin (IV/PO)), the glycopeptides (Vancomycin (IV), the lincosamides (Clindamycin (IV/PO), macrolides/ketolides (Azithromycin (IV/PO), Clarithromycin (PO), Dirithromycin, Erythromycin (IV/PO), Telithromycin), the cephalosporins (Cefadroxil (PO), Cefazolin (IV), Cephalexin (PO), Cephalothin, Cephapirin, Cephradine, Cefaclor (PO), Cefamandole (IV), Cefonicid, Cefotetan (IV), Cefoxitin (IV), Cefprozil (PO), Cefuroxime (IV/PO), Loracarbef (PO), Cefdinir (PO), Cefditoren (PO), Cefixime (PO), Cefoperazone (IV), Cefotaxime (IV), Cefpodoxime (PO), Ceftazidime (IV), Ceftibuten (PO), Ceftizoxime (IV), Ceftriaxone (IV), Cefepime (IV)), monobactams (Aztreonam (IV)), nitroimidazoles (Metronidazole (IV/PO)), oxazolidinones (Linezolid (IV/PO)), penicillins (Amoxicillin (PO), Amoxicillin/Clavulanate (PO), Ampicillin (IV/PO), Ampicillin/Sulbactam (IV), Bacampicillin (PO), Carbenicillin (PO), Cloxacillin, Dicloxacillin, Methicillin, Mezlocillin (IV), Nafcillin (IV), Oxacillin (IV), Penicillin G (IV), Penicillin V (PO), Piperacillin (IV), Piperacillin/Tazobactam (IV), Ticarcillin (IV), Ticarcillin/Clavulanate (IV)), streptogramins (Quinupristin/Dalfopristin (IV), sulfonamide/folate antagonists (Sulfamethoxazole/Trimethoprim (IV/PO)), tetracyclines (Demeclocycline, Doxycycline (IV/PO), Minocycline (IV/PO), Tetracycline (PO)), azole antifungals (Clotrimazole, Fluconazole (IV/PO), Itraconazole (IV/PO), Ketoconazole (PO), Miconazole, Voriconazole (IV/PO)), polyene antifungals (Amphotericin B (IV), Nystatin), echinocandin antifungals (Caspofungin (IV), Micafungin), and other antifungals (Ciclopirox, Flucytosine (PO), Griseofulvin (PO), Terbinafine (PO)).

D. Medical Devices

The invention also provides methods treat or prevent MRSA biofilms on medical devices composed of a wide variety of materials. Some examples of those materials include latex, latex silicone, silicone, polycarbonate, glass and polyvinyl chloride. Some examples of devices include endotracheal tubes, vascular catheters, including central venous catheters, arterial lines, pulmonary artery catheters, peripheral venous catheters, urinary catheters, nephrostomy tubes, stents such as biliary stents, peritoneal catheters, epidural catheters, naso-gastric and nasojejunal tubes, central nervous system catheters, including intraventricular shunts and devices, prosthetic valves, and sutures.

Another medical device that can be treated according to the present invention is an implant. Implants include artificial joints (hip, knee, elbow), pins, posts, plates, wires and rods, in particular those made of or containing titanium. About one million patients worldwide are treated annually for total replacement of arthritic hips and knee joints. The prostheses come in many shapes and sizes. Hip joints normally have a metallic femoral stem and head which locates into an ultrahigh molecular weight low friction polyethylene socket, both secured in position with polymethyl methacrylate bone cement.

Internal and external bone-fracture fixation provides a further major application for titanium as spinal fusion devices, pins, bone-plates, screws, intramedullary nails, and external fixators.

A major change in restorative dental practice worldwide has been possible through the use of titanium implants. A titanium ‘root’ is introduced into the jaw bone with time subsequently allowed for osseointegration. The superstructure of the tooth is then built onto the implant to give an effective replacement.

Surgery to repair facial damage using the patients own tissue cannot always obtain the desired results. Artificial parts may be required to restore the ability to speak or eat as well as for cosmetic appearance, to replace facial features lost through damage or disease. Osseointegrated titanium implants meeting all the requirements of biocompatibility and strength have made possible unprecedented advances in surgery, for the successful treatment of patients with large defects and hitherto highly problematic conditions.

Titanium is regularly used for pacemaker cases and defibrillators, as the carrier structure for replacement heart valves, and for intra-vascular stents. In addition, titanium is suitable for both temporary and long term external fixations and devices as well as for orthotic callipers and artificial limbs, both of which use titanium extensively for its light weight, toughness and corrosion resistance.

Thus, in one aspect, the invention comprises pre-treatment of devices prior to implant, thereby effectively reducing or preventing MRSA biofilm growth on the device once emplanted.

Alternatively, the device may be treated in vivo to prevent, limit, reduce or eliminate MRSA biofilms. As discussed above, the Agrquorum sensing agonists of the present invention may be used in combinations with antibiotics, and such is contemplated in the medical implant embodiment as well.

E. Non-Device Medical Infections

The present invention contemplates the treatment of medical infections not associated with indwelling medical devices. Several examples of such infections are discussed below.

i. Infectious Endocarditis

Endocarditis is an inflammation of the inner layer of the heart, the endocardium. It usually involves the heart valves (native or prosthetic valves). Other structures which may be involved include the interventricular septum, the chordae tendinae, the mural endocardium, or even on intracardiac devices. Endocarditis is characterized by a prototypic lesion, the “vegetation,” which is a mass of platelets, fibrin, microcolonies of microorganisms, and scant inflammatory cells. In the subacute form of infective endocarditis, the vegetation may also include a center of granulomatous tissue, which may fibrose or calcify.

Since the valves of the heart do not receive any dedicated blood supply, defensive immune mechanisms (such as white blood cells) cannot directly reach the valves via the bloodstream. If an organism (such as bacteria) attaches to a valve surface and forms a vegetation, the host immune response is blunted. The lack of blood supply to the valves also has implications on treatment, since drugs also have difficulty reaching the infected valve. Normally, blood flows smoothly through these valves. If they have been damaged (from rheumatic fever, for example) the risk of bacteria attachment is increased.

ii. Osteomyelitis

Osteomyelitis is an infection of bone or bone marrow, usually caused by pyogenic bacteria or mycobacteria. It can be usefully subclassified on the basis of the causative organism, the route, duration and anatomic location of the infection. Generally, microorganisms may infect bone through one or more of three basic methods: via the bloodstream, contiguously from local areas of infection (as in cellulitis), or penetrating trauma, including iatrogenic causes such as joint replacements or internal fixation of fractures or root-canaled teeth. Once the bone is infected, leukocytes enter the infected area, and in their attempt to engulf the infectious organisms, release enzymes that lyse the bone. Pus spreads into the bone's blood vessels, impairing their flow, and areas of devitalized infected bone, known as sequestra, form the basis of a chronic infection. Often, the body will try to create new bone around the area of necrosis. The resulting new bone is often called an involucrum. On histologic examination, these areas of necrotic bone are the basis for distinguishing between acute osteomyelitis and chronic osteomyelitis. Osteomyelitis is an infective process which encompasses all of the bone (osseous) components, including the bone marrow. When it is chronic, it can lead to bone sclerosis and deformity.

In infants, the infection can spread to the joint and cause arthritis. In children, large subperiosteal abscesses can form because the periosteum is loosely attached to the surface of the bone. Because of the particulars of their blood supply, the tibia, femur, humerus, vertebra, the maxilla, and the mandibular bodies are especially susceptible to osteomyelitis. However, abscesses of any bone may be precipitated by trauma to the affected area. Many infections are caused by Staphylococcus aureus.

Osteomyelitis often requires prolonged antibiotic therapy, with a course lasting a matter of weeks or months. A PICC line or central venous catheter is often placed for this purpose. Osteomyelitis also may require surgical debridement. Severe cases may lead to the loss of a limb. Initial first line antibiotic choice is determined by the patient's history and regional differences in common infective organisms. Hyperbaric oxygen therapy has been shown to be a useful adjunct to the treatment of refractory osteomyelitis. A treatment lasting 42 days is practiced in a number of facilities.

iii. Chronic Wounds

A chronic wound is a wound that does not heal in an orderly set of stages and in a predictable amount of time the way most wounds do; wounds that do not heal within three months are often considered chronic. Chronic wounds seem to be detained in one or more of the phases of wound healing. For example, chronic wounds often remain in the inflammatory stage for too long. In acute wounds, there is a precise balance between production and degradation of molecules such as collagen; in chronic wounds this balance is lost and degradation plays too large a role. Chronic wounds may never heal or may take years to do so. These wounds cause patients severe emotional and physical stress as well as creating a significant financial burden on patients and the whole healthcare system. Chronic wounds mostly affect people over the age of 60. The incidence is 0.78% of the population and the prevalence ranges from 0.18 to 0.32%. As the population ages, the number of chronic wounds is expected to rise. The vast majority of chronic wounds can be classified into three categories: venous ulcers, diabetic, and pressure ulcers. A small number of wounds that do not fall into these categories may be due to causes such as radiation poisoning or ischemia.

Venous ulcers, which usually occur in the legs, account for about 70% to 90% of chronic wounds and mostly affect the elderly. They are thought to be due to venous hypertension caused by improper function of valves that exist in the veins to prevent blood from flowing backward. Ischemia results from the dysfunction and, combined with reperfusion injury, causes the tissue damage that leads to the wounds.

Another major cause of chronic wounds, diabetes, is increasing in prevalence. Diabetics have a 15% higher risk for amputation than the general population due to chronic ulcers. Diabetes causes neuropathy, which inhibits nociception and the perception of pain. Thus patients may not initially notice small wounds to legs and feet, and may therefore fail to prevent infection or repeated injury. Further, diabetes causes immune compromise and damage to small blood vessels, preventing adequate oxygenation of tissue, which can cause chronic wounds. Pressure also plays a role in the formation of diabetic ulcers.

Another leading type of chronic wounds is pressure ulcers, which usually occur in people with conditions such as paralysis that inhibit movement of body parts that are commonly subjected to pressure such as the heels, shoulder blades, and sacrum. Pressure ulcers are caused by ischemia that occurs when pressure on the tissue is greater than the pressure in capillaries, and thus restricts blood flow into the area. Muscle tissue, which needs more oxygen and nutrients than skin does, shows the worst effects from prolonged pressure. As in other chronic ulcers, reperfusion injury damages tissue.

In addition to poor circulation, neuropathy, and difficulty moving, factors that contribute to chronic wounds include systemic illnesses, age, and repeated trauma. Comorbid ailments that may contribute to the formation of chronic wounds include vasculitis (an inflammation of blood vessels), immune suppression, pyoderma gangrenosum, and diseases that cause ischemia. Immune suppression can be caused by illnesses or medical drugs used over a long period, for example steroids. Emotional stress can also negatively affect the healing of a wound, possibly by raising blood pressure and levels of cortisol, which lowers immunity.

Though treatment of the different chronic wound types varies slightly, appropriate treatment seeks to address the problems at the root of chronic wounds, including ischemia, bacterial load, and imbalance of proteases. Various methods exist to ameliorate these problems, including antibiotic and antibacterial use, debridement, irrigation, vacuum-assisted closure, warming, oxygenation, moist wound healing, removing mechanical stress, and adding cells or other materials to secrete or enhance levels of healing factors.

To lower the bacterial count in wounds, therapists may use topical antibiotics, which kill bacteria and can also help by keeping the wound environment moist, which is important for speeding the healing of chronic wounds. Some researchers have experimented with the use of tea tree oil, an antibacterial agent which also has anti-inflammatory effects. Disinfectants are contraindicated because they damage tissues and delay wound contraction. Further, they are rendered ineffective by organic matter in wounds like blood and exudate and are thus not useful in open wounds.

A greater amount of exudate and necrotic tissue in a wound increases likelihood of infection by serving as a medium for bacterial growth away from the host's defenses. Since bacteria thrive on dead tissue, wounds are often surgically debrided to remove the devitalized tissue. Debridement and drainage of wound fluid are an especially important part of the treatment for diabetic ulcers, which may create the need for amputation if infection gets out of control. Mechanical removal of bacteria and devitalized tissue is also the idea behind wound irrigation, which is accomplished using pulsed lavage.

Removing necrotic or devitalzed tissue is also the aim of maggot therapy, the intentional introduction by a health care practitioner of live, disinfected maggots non-healing wounds. Maggots dissolve only necrotic, infected tissue; disinfect the wound by killing bacteria; and stimulate wound healing. Maggot therapy has been shown to accelerate debridement of necrotic wounds and reduce the bacterial load of the wound, leading to earlier healing, reduced wound odor and less pain. The combination and interactions of these actions make maggots an extremely potent tool in chronic wound care.

Negative pressure wound therapy (NPWT) is a treatment that improves ischemic tissues and removes wound fluid used by bacteria. This therapy, also known as vacuum-assisted closure, reduces swelling in tissues, which brings more blood and nutrients to the area, as does the negative pressure itself. The treatment also decompresses tissues and alters the shape of cells, causes them to express different mRNAs and to proliferate and produce ECM molecules.

### IV. EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Strains and growth conditions. All DNA manipulations were performed in Escherichia coli DH5α-E (Invitrogen, Carlsbad, Calif.). Strains of E. coli were grown in Luria-Bertani broth (LB), and growth medium was supplemented with ampicillin (100 ug/ml) for plasmids. Staphylococcus aureus strains used were: RN4220; CA-MRSA isolate LAC (Voyich et al., 2005); AH1203, ΔAgr::Tet derivative of strain LAC (Lauderdale et al., 2009). Strains of S. aureus were grown in tryptic soy broth (TSB). For maintenance of plasmids in S. aureus, antibiotic concentrations were (in μg/ml): chloramphenicol (Cam), 10; spectinomycin (Spc), 1000. All reagents were purchased from Fisher Scientific (Pittsburg, Pa.) and Sigma (St. Louis, Mo.) unless otherwise indicated.

Genetic techniques. Plasmids were transformed into S. aureus RN4220 by electroporation as previously described (Lauderdale et al., 2009). Plasmids were moved from strain RN4220 to other strains using transduction by bacteriophage α80 as previously described (Novick, 1991).

Biofilm Assays. For biofilms grown on glass, flow cell biofilm growth, confocal scanning laser microscopy (CSLM), and computer image analysis were performed as previously described (Boles and Horswill, 2008). To monitor biofilm growth, LAC was transformed with GFP-expressing plasmid pALC2084 (Bateman et al., 2001), and the growth medium was supplemented with 1 μg/ml Cam and 20 ng/ml anhydrotetracycline for plasmid maintenance and induction of GFP expression. For the titanium biofilms, a BioSurface FC270 dual channel flow cell (BioSurface Technologies Corp., Bozeman, Mont.) was adapted. The polycarbonate coupons were replaced with titanium disks of the identical size, cut from a corrosion resistant, pure titanium rod (grade 2, McMaster-Carr, Atlanta, Ga.). The titanium disks were generated at the University of Iowa Medical Instruments Facility. For the titanium biofilms, flow cell, media and methods were as previously described (Boles and Horswill, 2008), except that the flow rate was 0.5 ml/min (10 rpm) on a Watson Marlow peristaltic pump (model 205S). To GFP label cells, plasmid pCM12 was utilized, and this plasmid constitutively expresses GFP (plasmid details to be published elsewhere). The flow cell growth medium was supplemented with 100 μg/ml Spc for pCM12 plasmid maintenance. The computational analysis program COMSTAT was employed to obtain measurements of biofilm properties (Heydorn et al., 2000). For biofilm dispersal tests, AIP type I (AIP-I) was synthesized and quantified using an intein system (Malone et al., 2007), and biofilm dispersal tests were performed as previously described (Boles and Horswill, 2008). For testing biofilm properties, Proteinase K (Research Productions International) was added to a final concentration of 2 μg/mL, and DNaseI (bovine pancreas, RNase free, Qiagen) was added to a final concentration of 0.5 Units/mL. LAC biofilm integrity was monitored at 6 hr and 22 hr, and COMSTAT analysis was performed to quantify the biofilm properties.

Antibiotic susceptibility. Titantium biofilms of LAC were grown, and at 72 hr post-inoculation, the chambers were disassembled under sterile conditions. Disks were gently placed in 1 mL phosphate buffered saline (PBS) containing levofloxacin and rifamipicin at the indicated concentrations and incubated statically for 6 hr at room temperature. After incubation, disks were washed in 1 mL PBS, sonicated for 10 min using a Branson 1200 water bath, and dilutions were plated to quantify colony forming units (CFU). To determine antibiotic resistance of dispersed biofilms, AIP was added (˜50 nM) to the media reservoir after 48 hr growth, and the biofilm effluent was collected on ice for 24 hr. Cell concentration was adjusted to the equivalent of one coupon of biomass, or ˜1×10⁸ CFU. Cells were treated as described above with the exception that cells were pelleted by centrifugation at 13,000 RPM and then resuspended in 1 mL PBS before sonication. To determine antibiotic resistance of planktonically grown cells, an overnight culture of LAC was grown in TSB with 0.2% glucose, and the optical density of the overnight culture was determined. For the resistance test, ˜1×10⁸ cells were collected and treated identically to the AIP-dispersed cells.

### Example 2

### Results

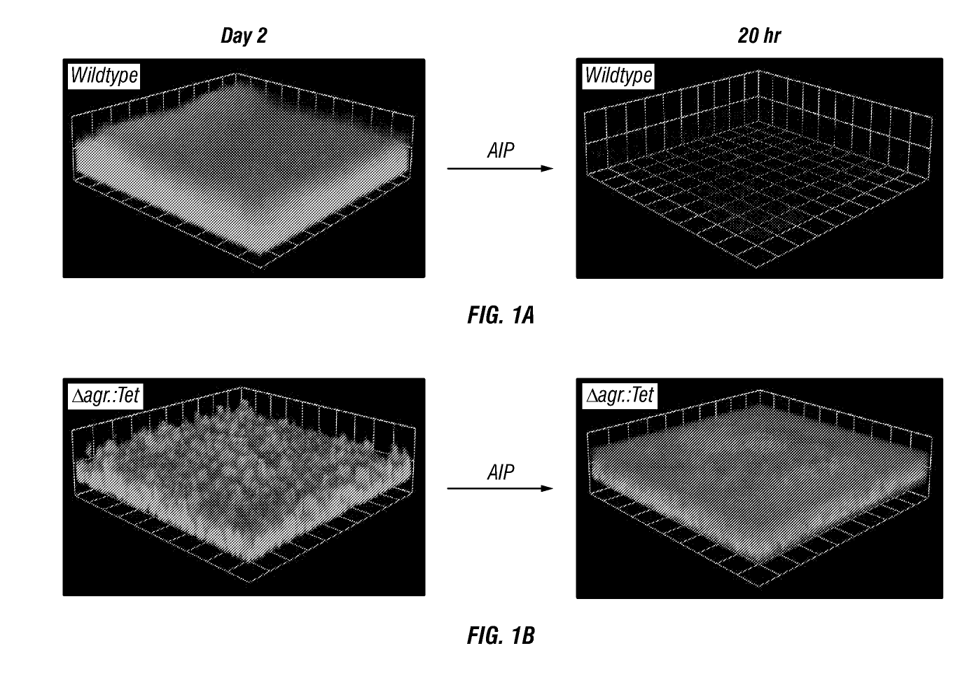

USA300 has a functional biofilm dispersal pathway. The inventor investigated biofilm formation using the CA-MRSA clinical isolate “LAC,” which is a member of the USA300-0114 strain lineage (Voyich et al., 2005). The LAC strain has become the USA300 model of choice for many in vitro and in vivo studies, and based on this precedent, the inventor utilized LAC to gain insight on USA300 biofilm maturation and dispersal. Recently, the inventor demonstrated that strain LAC is a strong biofilm former on a glass surface using a once-through, continuous flow cell apparatus (Lauderdale et al., 2009). To further investigate biofilm development, the inventor tested whether the LAC strain has a functional Agr-dispersal mechanism. Established LAC biofilms were prepared and cells were labeled with GFP using plasmid pALC2084 (Bateman et al., 2001). Biofilm integrity was monitored using confocal laser scanning microscopy (CLSM), and as anticipated, the LAC biofilm appeared as a confluent layer ˜20 μm thick (FIG. 1A). After 48 hr of growth, the quorum-sensing signal AIP type I (AIP-I) was added to a final concentration of ˜50 nm to the biofilm media. The biofilm integrity was monitored at 20 hr following signal addition, and the inventor observed a complete loss of biomass (FIG. 1A). In scanning the glass substratum, remaining live cells could not be detected, indicating the biomass completely dispersed from the surface. Similar to a previous report from the inventor (Boles and Horswill, 2008), when the Agrquorum-sensing system was inactivated through mutation, the addition of the inducing AIP-I signal had no effect on the biofilm integrity (FIG. 1B). These initial observations demonstrate that USA300 biofilms have a functional quorum-sensing dispersal pathway.

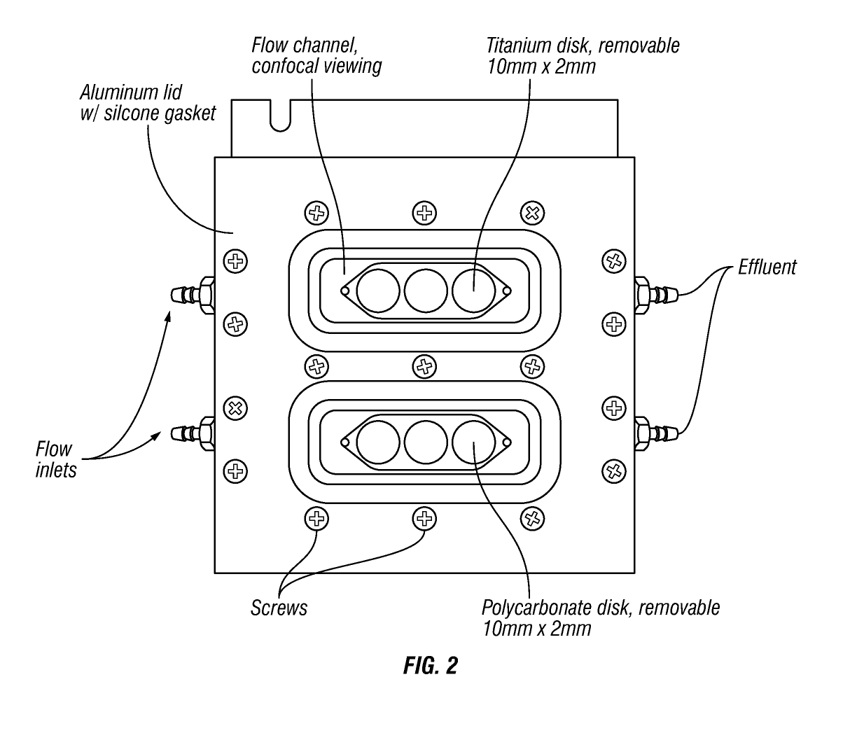

Flow cell apparatus for growing titanium biofilms. The inventor has adapted a flow cell apparatus for growing S. aureus biofilms on orthopaedic implant materials. The base chamber is a BioSurface Technologies flow cell FC251, which is a dual channel flow cell equipped with three removable polycarbonate coupons (FIG. 2). The removal coupons are a convenient size (10 mm×2 mm) that allows other surfaces to be examined in this apparatus. Titanium is a frequent material used in orthopaedic implants and made for a starting point for testing alternative, potentially more relevant abiotic surfaces as an indicator of foreign body infection. A pure titanium rod was obtained and disks were cut and machined to exactly match the size of the polycarbonate coupons (FIG. 2).

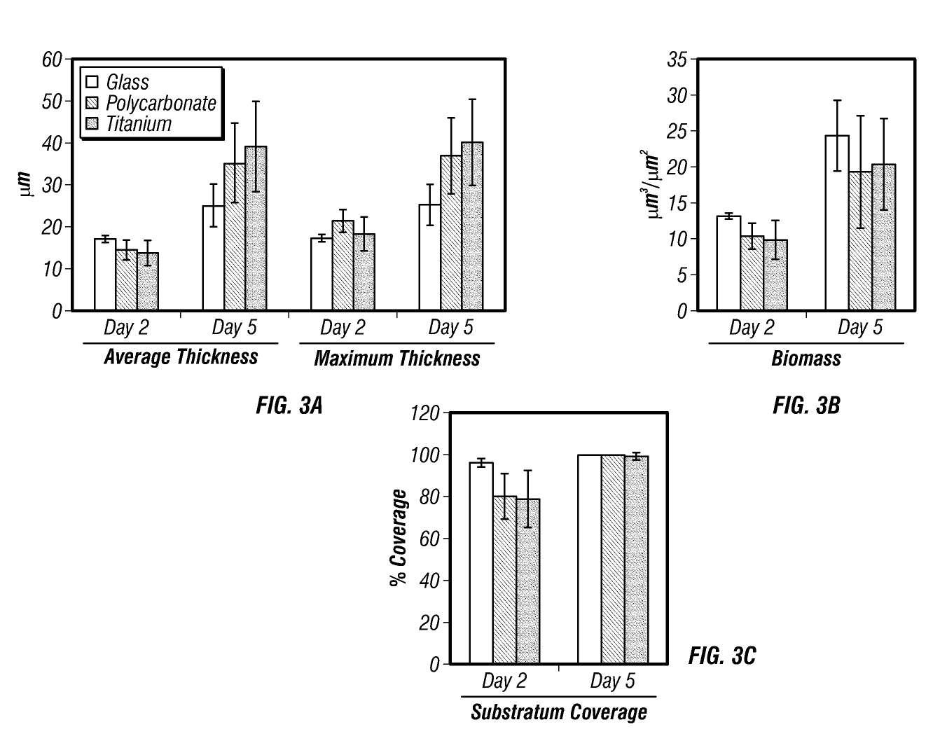

Biofilm maturation on titanium compared to other surfaces. To compare biofilm growth on different abiotic surfaces, LAC biofilms were prepared in the new flow cell chamber with alternating titanium and polycarbonate coupons (FIG. 2). COMSTAT analysis was performed to compare notable features of the biofilms grown on the different surface chemistries. At 2 days growth, the average biofilm thickness was 13.4 μm on titanium versus 14.4 μm on polycarbonate (FIG. 3A), and maximum thickness on the same surfaces was 18.1 μm versus 21.3 μm, respectively. At 5 days, the biofilm grew to 39 um average thickness on titanium versus 35.2 μm on polycarbonate. Factoring in error across multiple flow cell runs and assembled images, there was no significant difference between the titanium and polycarbonate surfaces. The amount of total biomass (μm³/μm²) paralleled this trend with low biomass at day 2 and substantially more at day 5 (FIG. 3B), and again little difference between the titanium and polycarbonate surfaces. These results were also compared to LAC biofilms grown on a glass surface. At 2 days, the glass grown biofilms were very similar to the other surfaces, but at 5 days, the glass biofilms were somewhat thinner while at the same time exhibiting denser biomass (FIG. 3B). Surface coverage was also assessed across the three different surfaces. At 2 days of growth, 80% of the titanium and polycarbonate surface was covered, which increased to 100% at 5 days (FIG. 3C). Again, there was no difference between these two surfaces in this indicator of biofilm maturation. With the alternative glass surface, LAC displayed improved surface coverage at 2 days of 96%. Overall, the analysis of various biofilm properties at different time points indicates the LAC strain displays surprisingly little variation in biofilm maturation on three different abiotic surfaces.

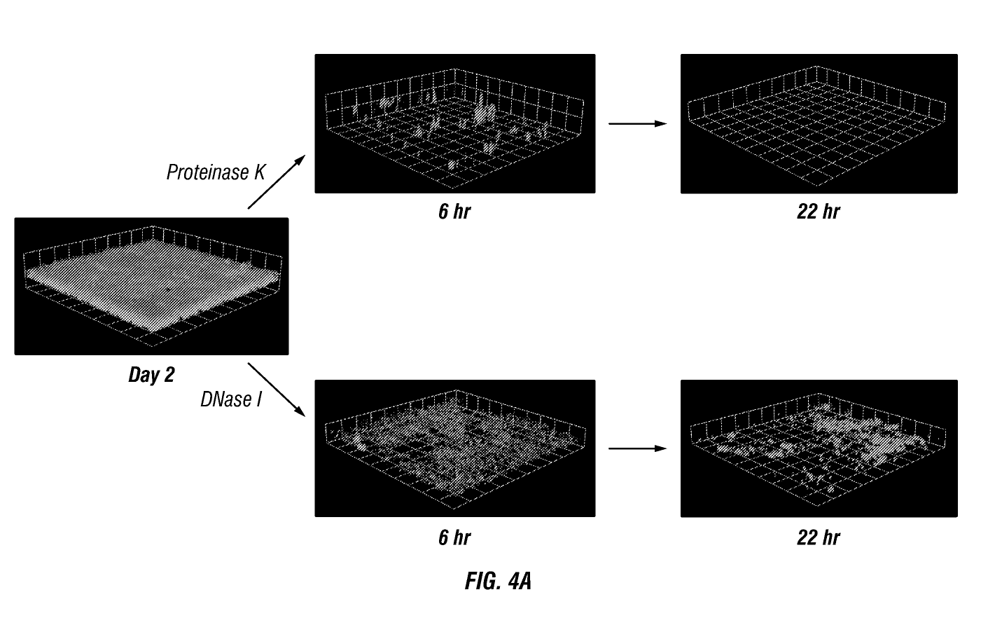

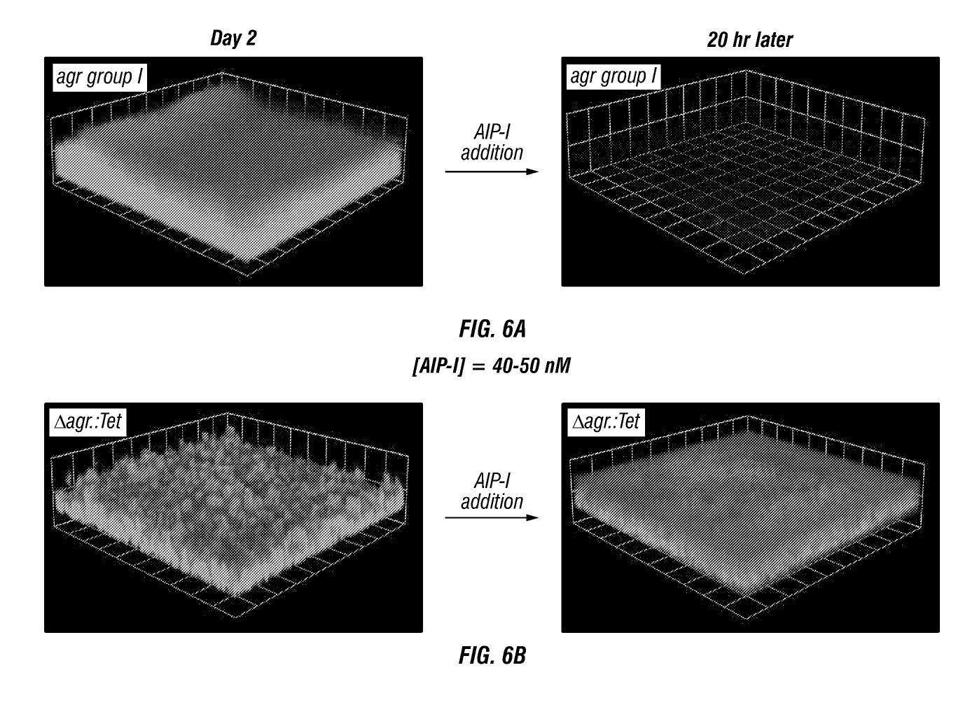

Properties of titanium biofilms. To determine whether Agr-mediated biofilm dispersal operates on titanium, two day LAC biofilms were grown. Similar to the glass surface, the inventor observed complete dispersal of the biomass at 20 hr post AIP-I addition (FIG. 6A; SFIG. 1A). Again, when the Agrquorum-sensing system was inactivated, the LAC biofilm did not respond to AIP-I treatment (FIG. 6B; SFIG. 1B). Recent studies in the inventors' laboratory also indicate that strain LAC does not require exopolysaccharide to develop a mature biofilm (Lauderdale et al., 2009), which parallels results obtained with other MRSA isolates (O'Neill et al., 2007). In the absence of exopolysaccharide, many S. aureus strains develop a biofilm matrix predominantly composed of proteinaceous material and extracellular DNA (eDNA) (Boles and Horswill, 2008; Rice et al., 2007). To investigate properties of the LAC biofilm matrix, titanium biofilms were grown for 2 days and treated with either proteinase K or DNaseI (bovine pancreas). Following proteinase K treatment (2 μg/mL), approximately 93% of LAC biomass detached from the titanium surface in 6 hr (FIGS. 4A-B) and at 22 hr, all of the LAC biomass had detached. In a parallel test with DNaseI treatment (0.5 Units/mL), 35% of the LAC biomass detached in 6 hr, and by 22 hr, 85% of the biomass had detached. The remaining biofilm is evident in the confocal image as microcolonies on the titanium surface, and at least in this time frame, this biomass was resistant to DNaseI exposure.

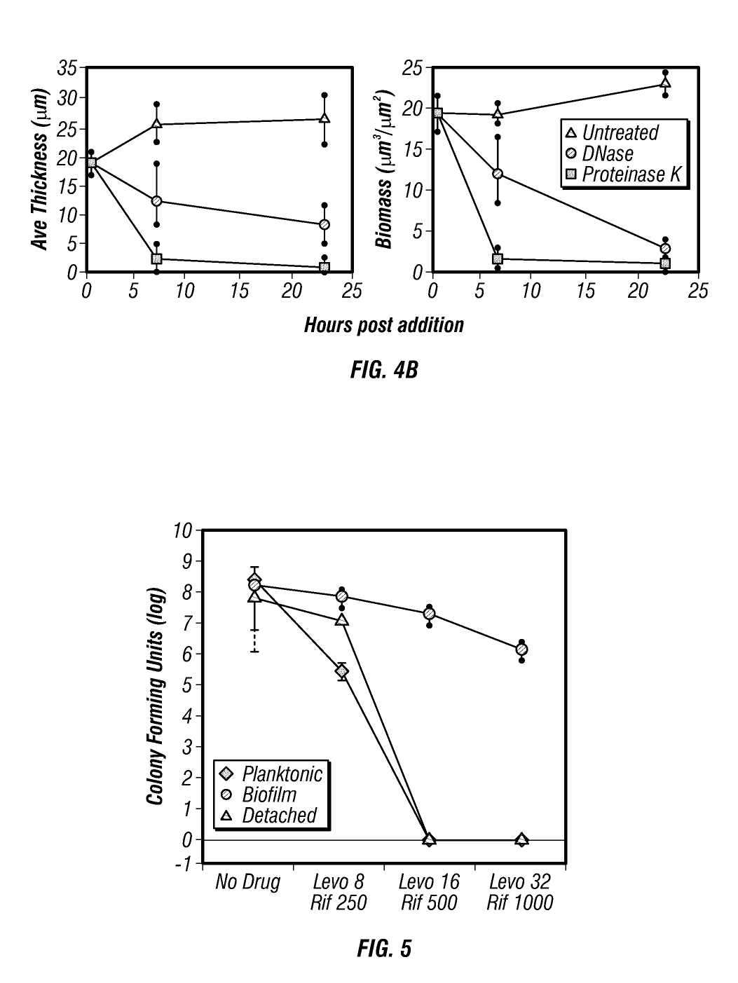

Biofilm dispersal restores antibiotic susceptibility. One of the defining features of biofilms is the enhanced resistance to antimicrobial treatment (Costerton, 2005). Using an methicillin-susceptible S. aureus (MSSA) stain, the inventor previously demonstrated that AIP-mediated biofilm dispersal could restore antibiotic susceptibility (Boles and Horswill, 2008). To test LAC biofilms on titanium, biofilms were grown for 72 hr and resistance to rifampicin and levofloxacin was tested. As anticipated, the established biofilms displayed potent antibiotic resistance (FIG. 5), and only the highest concentrations of rifampicin (1000 μg/mL) and levofloxacin (32 μg/mL) resulted in loss of greater than 1 log of viable cells. To test antibiotic susceptibility of dispersed cells, LAC biofilms were grown for 48 hr on titanium, exposed to AIP (˜50 nM) for 24 hr, and detached cells were collected from the effluent. The AIP-dispersed cells were significantly more antibiotic susceptible with a greater than 7-log difference at rifampicin (500 μg/mL) and levofloxacin (16 μg/mL) and complete killing at higher antibiotic concentrations. As a control, planktonic cells were tested using broth culture, and notably, the antibiotic resistance trend of the broth culture mirrored that of the detached cells. These results support the inventor's previous observations and demonstrate that AIP-mediated dispersal restores antibiotic susceptibility to both MSSA and MRSA biofilms.

### Example 3

### Discussion

In this study, the inventor provides the first demonstration that MRSA strains possess a functional biofilm dispersal pathway. Importantly, this mechanism was identified using an CA-MRSA USA300 isolate and shown to function on a titanium surface, a common material used in orthopaedic implants. The properties of the USA300 biofilm on titanium were also examined and the biofilm matrix was found to be composed of proteinaceous material and eDNA. When these biofilms were dispersed from the titanium surface, the cells regained antibiotic susceptibility, suggesting exogenous control of the dispersal mechanism could be an effective therapeutic treatment for biofilm infections.

The results with the USA300 biofilm dispersal test paralleled our observations with MSSA strains (Boles and Horswill, 2008). As long as the Agrquorum-sensing system is functional, the exogenous addition of the AIP signal dispersed the biofilm. Interestingly, the activation kinetics of biofilm dispersal and effectiveness of the mechanism was substantially more robust in USA300 compared to MSSA strains (Boles and Horswill, 2008). Notably, the USA300 biofilm dispersed in a shorter time frame and more completely in comparison to MSSA isolate SH1000. In part, the larger dynamic range of the Agrsystem in LAC and other USA300 isolates might contribute to the improved kinetics and robustness of the dispersal phenotype (Wang et al., 2007).

One surprising finding in this study was the lack of changes in biofilm characteristics using different surface chemistries. Comparing glass, polycarbonate, and titanium surfaces, there was no significant difference in USA300 biofilm maturation in terms of film thickness, total biomass, or surface coverage. At early time points, the biofilm displayed an improved ability to coat the glass surface, but this change was negligible at later time points. Importantly, biofilm dispersal also functioned in a related manner on each surface. Thus, USA300 can establish similar biofilms on diverse abiotic surfaces, which could be an important factor in implant infections.

The presence of proteins and eDNA in the MRSA biofilm matrix parallels recent reports with other S. aureus strains (O'Neill et al., 2007; Rice et al., 2007). The Agrquorum-sensing system is known to induce the expression of extracellular protease activity and a secreted DNase (Novick and Geisinger, 2008), and while the dispersal mechanism remains unresolved, the combined action of these enzymes could be contributing to the dispersal phenotype (Boles and Horswill, 2008). Other Agrregulated factors, such as the phenol soluble modulins (Vuong et al., 2000), have also been linked to biofilm dispersal. Further studies are necessary to clarify the factors necessary for the dispersal mechanism. Considering the high rate of morbidity and mortality in MRSA infections, the results of this study show great promise for the development of innovative therapies that could greatly improve the final outcomes of technically successful orthopaedic surgeries and reconstructions.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,415,732
U.S. Pat. No. 4,458,066,
U.S. Pat. No. 4,946,778
U.S. Pat. No. 5,354,855
U.S. Pat. No. 5,795,715
U.S. Pat. No. 5,888,773
U.S. Pat. No. 5,889,136
U.S. Pat. No. 6,337,385
U.S. Pat. No. 6,953,833
U.S. Patent Publication 2007/0185016
Allison et al., FEMS Microbiol. Lett., 167:179-184, 1998.
Atkinson et al., Mol. Microbiol., 33:1267-1277, 1999.
Ayliffe, Clin. Infect. Dis., 24:S74-9, 1997.
Baba et al., Lancet., 359(9320):1819-1827, 2002.
Banin et al., Appl. Environ. Microbiol., 72:2064-2069, 2006.
Barber, J. Clin. Pathol., 14:385-393, 1961.
Barberan, Clin. Microbiol. Infect., 12(3):93-101, 2006.
Barraud et al., J. Bacteriol., 188:7344-7353, 2006.
Bassetti et al., J. Antimicrob. Chemother., 55:387-90, 2005.
Bateman et al., Infect. Immun., 69:7851-7, 2001.
Beenken et al., Infect. Immun., 71:4206-4211, 2003.
Begier et al., Clin Infect Dis., 39(10):1446-1453, 2004.
Beilman et al., Surg Infect (Larchmt)., 6(1):87-92, 2005.
Bischoff et al., J. Bacteriol., 186:4085-4099, 2004.
Boles and Horswill, PLoS Pathogens, 4:e1000053, 2008.
Boles et al., Mol. Microbiol., 57:1210-1223, 2005.
Bosher and Labouesse, Nat. Cell. Biol., 2(2):E31-E36, 2000.
Bratu et al., Emerg. Infect. Dis., 11(6):808-813, 2005.
Capaldi et al., Biochem. Biophys. Res. Comm., 74(2):425-433, 1977.
Caplen et al., Gene, 252(1-2):95-105, 2000.
Chaignon et al., Appl. Microbiol. Biotechnol., 75:125-132, 2007.
Chalumeau et al., Clin. Infect. Dis., 41(3):e29-30, 2005.
Cheung et al., Infect. Immun., 67:1331-1337, 1999.
Conly et al., Can. J. Infect. Dis. Med. Microbiol., 16:109, 2005.
Cook et al., Cell, 27:487-496, 1981.
Costerton, Clin. Orthop. Relat. Res., 7-11, 2005.
Cramton et al., Infect. Immun., 67:5427-5433, 1999.
Crossley et al., J. Infect. Dis., 139:273-279, 1979.
Davey and O'Toole, Microbiol. Mol. Biol. Rev., 64:847-867, 2000.
Davey et al., J. Bacteriol., 185:1027-1036, 2003.
Davies et al., Science, 280:295-298, 1998.
Davies, Nat. Rev. Drug Discov., 2:114-122, 2003.
Davis et al., J. Biol. Chem., 252:6544-6553, 1977.
Diep et al., The Lancet, 367:731-739, 2006.
Dow et al., Proc. Natl. Acad. Sci. USA, 100:10995-11000, 2003.
Elbashir et al., EMBO, 20(23):6877-6888, 2001.
Elbashir et al., Genes Dev., 5(2):188-200, 2001.
Elbashir et al., Nature, 411(6836):494-498, 2001.
Entenza et al., Infect. Immun., 73(2):990-998, 2005.
Fire et al., Nature, 391(6669):806-811, 1998.
Fleming et al., J. Bacteriol., 188:7686-7688, 2006.
Forster and Symons, Cell, 48:211-220, 1987.
Francis et al., Clin. Infect. Dis., 40(1):100-107, 2005.
Furukawa et al., J. Bacteriol., 188:1211-1217, 2006.
Fux et al., J. Bacteriol., 186:4486-4491, 2004.
Gerlach et al., Nature (London), 328:802-805, 1987.
Gertz et al., Mol. Gen. Genet., 261:558-566, 1999.
Gilbert et al., Can. J. Infect. Dis. Med. Microbiol., 16:108, 2005.
Gilbert et al., CMAJ, 175(2):149-154, 2006.
Grishok et al., Science, 287:2494-2497, 2000.
Hackbarth & Chambers, Antimicrob. Agents Chemother., 33(7):995-999, 1989.
Hall-Stoodley and Stoodley, Trends Microbiol., 13:7-10, 2005.
Hague et al., Int. J. Antimicrob. Agents, 30:72-7, 2007.
Harbarth et al., Emerg. Infect. Dis., 11(6):962-965, 2005.
Harris and Richards, Injury, 37(Suppl 2):S3-14, 2006.
Heydorn et al., Microbiology, 146(Pt 10):2395-2407, 2000.
Heydorn et al., Microbiology, 146(Pt 10):2395-407, 2000.
Holmes et al., J. Clin. Microbiol., 43(5):2384-2390, 2005.
Horsburgh et al., J. Bacteriol., 184:5457-5467, 2002.
Huber et al., Microbiology, 147:2517-2528, 2001.
Hunt et al., Appl. Environ. Microbiol., 70:7418-7425, 2004.
Irie et al., FEMS Microbiol. Lett., 250:237-243, 2005.
Issartel et al., Clin. Microbiol., 43(7):3203-3207, 2005.
Ito et al., Antimicrob. Agents Chemother., 45:1323-1336, 2001.
Ito et al., Antimicrob. Agents Chemother., 48:2637-2651, 2004.
Izano et al., Appl. Environ. Microbiol., 74:470-476, 2008.
Jarraud et al., Infect. Immun., 70:631-641, 2002.
Jarraud et al., J. Bacteriol., 182:6517-6522, 2000.
Jevons, British Med. J., 1:124-125, 1961.
Ji et al., Science, 276:2027-2030, 1997.
Joyce, Nature, 338:217-244, 1989.
Kaplan et al., J. Bacteriol., 185:4693-4698, 2003.
Kaplan et al., J. Bacteriol., 186:8213-8220, 2004.
Kavanaugh et al., Mol. Microbiol., 65:780-798, 2007.
Kazakova et al., N. Engl. J. Med., 352(5):468-475, 2005.
Ketting et al., Cell, 99(2):133-141, 1999.
Kim and Cook, Proc. Natl. Acad. Sci. USA, 84(24):8788-8792, 1987.
Klevens et al., JAMA, 298:1763-71, 2007.
Knobloch et al., Infect. Immun., 72:3838-3848, 2004.
Knobloch et al., J. Bacteriol., 183:2624-2633, 2001.
Kourbatova et al., Am. J. Infect. Control, 33:385-91, 2005.
Kullik and Giachino, Arch. Microbiol., 167:151-159, 1997.
Lauderdale et al., Infect. Immun., 77:1623-35, 2009.
Leid et al., Infect. Immun., 70:6339-6345, 2002.
Lin et al., J. Gen. Virol., 80:91-96, 1999.
Linde et al., Eur. J. Clin. Microbiol. Infect. Dis., 24(6):419-422, 2005.
Lindsay and Holden, Trends Microbiol., 12:378-385, 2004.
Livermore, Int. J. Antimicrob. Agents, 16(1:)S3-10, 2000.
Lodise, Jr. and McKinnon, Pharmacotherapy, 27:1001-12, 2007.
Lyon et al., Biochemistry, 41:10095-10104, 2002.
Ma et al., Antimicrob. Agents Chemother., 46:1147-1152, 2002.
Malone et al., Appl. Environ. Microbiol., 73(9):6036-6044, 2007.
Malone et al., Appl. Environ. Microbiol., 73:6036-44, 2007.
Marcotte and Trzeciak, J. Am. Acad. Orthop. Surg., 16:98-106, 2008.
Mayville et al., Proc. Natl. Acad. Sci. USA, 96:1218-1223, 1999.
Michel and Westhof, J. Mol. Biol., 216:585-610, 1990.
Miller and Diep, Clin. Infect. Dis., 46:752-60, 2008.
Montgomery et al., Proc. Natl. Acad. Sci. USA, 95:15502-15507, 1998.
Morgan et al., J. Bacteriol., 188:7335-7343, 2006.
Mulvey et al., Emerg. Infect. Dis., 11(6):844-850, 2005.
Musk et al., Chem. Biol., 12:789-796, 2005.
Naas et al., J. Hosp. Infect., 61(4):321-329, 2005.
Novick and Geisinger, Annu. Rev. Genet., 42:541-64, 2008.
Novick, Methods Enzymol., 204:587-636, 1991.
Novick, Mol. Microbiol., 48:1429-1449, 2003.
O'Gara, FEMS Microbiol. Lett., 270:179-188, 2007.
O'Neill et al., J. Clin. Microbiol., 45:1379-1388, 2007.
O'Neill et al., J. Clin. Microbiol., 45:1379-88, 2007.
Pan et al., Clin. Infect. Dis., 37(10):1384-1388, 2003.
Pane-Farre et al., Int. J. Med. Microbiol., 296:237-258, 2006.
Panlilio et al., Infect. Control Hosp. Epidemiol., 13:582-586, 1992.
Parsek and Singh, Annu. Rev. Microbiol., 57:677-701, 2003.
Patel, Clin. Orthop. Relat. Res., 41-47, 2005.
PCT Appln. PCT US2007/087663
PCT Appln. WO 00/44914
PCT Appln. WO 01/68836
PCT Appln. WO 84/03564
PCT Appln. WO 99/32619
PCT Appln. WO 01/36646
Puskas et al., J. Bacteriol., 179:7530-7537, 1997.
Qiu et al., J. Biol. Chem., 280:16695-16704, 2005.
Rachid et al., J. Bacteriol., 192:6824-6826, 2000.
Reinhold-Hurek and Shub, Nature, 357:173-176, 1992.
Rice et al., J. Bacteriol., 187:3477-3485, 2005.
Rice et al., Proc. Natl. Acad. Sci. USA, 104:8113-8, 2007.
Rice et al., Proc. Natl. Acad. Sci. USA, 104:8113-8118, 2007.
Robert et al., Clin. Microbiol. Infect., 11(7):585-587, 2005.
Rohde et al., Biomaterials, 28:1711-1720, 2007.
Said-Salim et al., J. Clin. Microbiol., 43(7):3373-3379, 2005.
Sarver et al., Science, 247:1222-1225, 1990.
Sauer et al., J. Bacteriol., 186:7312-7326, 2004.
Scanlon et al., Proc. Natl. Acad. Sci. USA, 88:10591-10595, 1991.
Segawa et al., J. Bone Joint Surg., 81 :1434-45, 1999.
Seybold et al., Infection, 35:190-3, 2007.
Sharp and Zamore, Science, 287:2431-2433, 2000.
Sharp, Genes Dev., 13:139-141, 1999.
Stoodley et al., J. Bone Joint Surg. Am., 90:1751-8, 2008.
Sung et al., Infect. Immun., 74:2947-2956, 2006.
Tabara et al., Cell, 99(2):123-132, 1999.
Tenover et al., J. Clin. Microbiol., 44(1):108-118, 2006.
Thormann et al., J. Bacteriol., 187:1014-1021, 2005.
Thormann et al., J. Bacteriol., 188:2681-2691, 2006.
Toledo-Arana et al., J. Bacteriol., 187:5318-5329, 2005.
Valle et al., Mol. Microbiol., 48:1075-1087, 2003.
Vandenesch et al., Emerg. Infect. Dis., 9(8):978-984, 2003.
Vandenesch et al., Emerg. Infect. Dis., 9(8):978-984, 2003.
Voss et al., Eur. J. Clin. Microbiol. Infect. Dis., 13:50-55, 1994.
Vourli et al., Euro. Surveill., 10(5):78-79, 2005.
Voyich et al., J. Immunol., 175:3907-19, 2005.
Vuong et al., J. Infect. Dis., 182:1688-1693, 2000.
Wang et al., Nat. Med., 13:1510-1514, 2007.
Wannet et al., J. Clin. Microbiol., 42(7):3077-3082, 2004.
Wannet et al., J. Clin. Microbiol., 43(7):3341-3345, 2005.
Whitchurch et al., Science, 295:1487, 2002.
Wincott et al., Nucleic Acids Res., 23(14):2677-2684, 1995.
Witte et al., Eur. J. Clin. Microbiol. Infect. Dis., 24(1):1-5, 2005.
Wright et al., Proc. Natl. Acad. Sci. USA, 101:16168-161-73, 2004.
Wright et al., Proc. Natl. Acad. Sci. USA, 102:1691-1696, 2005.
Wylie and Nowicki, J. Clin. Microbiol., 43(6):2830-2836, 2005.
Yarwood et al., J. Bacteriol., 186:1838-1850, 2004.
Zhang and Ji, J. Bacteriol., 186:6706-6713, 2004.
Zhang and Pierson, Appl. Environ. Microbiol., 67:4305-4315, 2001.
Ziebandt et al., Proteomics, 1:480-493, 2001.
Ziebandt et al., Proteomics, 4:3034-3047, 2004.
Zimmerli et al., N. Engl. J. Med., 351:1645-54, 2004.

1. A method of inhibiting a methicillin resistant Staphylococcus aureus (MRSA) biofilm formation comprising contacting a biofilm-forming MRSA with an activator of an Agrquorum-sensing system.

2. The method of claim 1, wherein said Agrquorum-sensing system is Agr-I.

3. The method of claim 1, wherein said Agrquorum-sensing system is Agr-II.

4. The method of claim 1, wherein said Agrquorum-sensing system is Agr-III.

5. The method of claim 1, wherein said Agrquorum-sensing system is Agr-IV.

6. The method of claim 1, wherein said activator is an autoinducing peptide (AIP).

7. The method of claim 1, further comprising contacting said MRSA with an antibiotic or antiseptic agent.

8. The method of claim 1, wherein inhibiting comprises inhibiting biofilm formation.

9. The method of claim 1, wherein inhibiting comprises inhibiting biofilm growth.

10. The method of claim 1, wherein inhibiting comprises reducing biofilm size.

11. The method of claim 1, wherein inhibiting comprises promoting detachment of MRSA from a formed biofilm.

12. The method of claim 1, wherein said MRSA biofilm or biofilm-forming MRSA is located in a subject.

13. The method of claim 12, wherein said subject is a mammalian subject.

14. The method of claim 13, wherein said mammalian subject is a human subject.

15. The method of claim 12, wherein said subject comprises an in-dwelling medical device or implant.

16. The method of claim 15, wherein said in-dwelling medical device is a catheter, a pump, endotracheal tube, a nephrostomy tube, a stent, an orthopedic device, or a suture, or a prosthetic valve.

17. The method of claim 15, wherein said catheter is a vascular catheter, an urinary catheter, a peritoneal catheter, an epidural catheter, a central nervous system catheter, central venous catheter, an arterial line catheter, a pulmonary artery catheter, or a peripheral venous catheter.

18. The method of claim 12, wherein said MRSA biofilm or biofilm-forming MRSA is located on a wound dressing.

19. The method of claim 12, wherein said MRSA biofilm or biofilm-forming MRSA is located on a tissue surface.

20. The method of claim 18 wherein said tissue surface is a heart valve, bone or epithelia.

21. The method of claim 1, wherein said MRSA biofilm or biofilm-forming bacterium is located on an inanimate surface.

22. The method of claim 17 wherein said inanimate surface is a floor, a table-top, a counter-top, a medical device surface, a wheelchair surface, a bed surface, a sink, a toilet, a filter, a valve, a coupling, or a tank.

23. The method of claim 15, further comprising coating said in-dwelling medical device with said inhibitor prior to implantation.

24. The method of claim 1, wherein said inhibitor is a SigB inhibitor.

25. A method of preventing methicillin resistant Staphylococcus aureus (MRSA) biofilm formation secondary to nosocomial infection in a subject comprising administering to said subject an activator of an Agrquorum-sensing system in combination with an antibiotic

26. The method of claim 25, wherein said nosocomial infection is pneumonia, bacteremia, a urinary tract infection, a catheter-exit site infection, and a surgical wound infection.

27. A method of restoring antibiotic sensitivity to a methicillin resistant Staphylococcus aureus (MRSA) located in a biofilm comprising contacting said MRSA with an activator of an Agrquorum-sensing system.

28. The method of claim 27, further comprising administerting to said subject an antibiotic.


SEQUENCE LISTING (4 sequences)

SEQ ID NO: 1 - protein, 8 aa, Synechocystis PCC6803
YSTCDFIM

SEQ ID NO: 2 - protein, 9 aa, Synechocystis PCC6803
GVNACSSLF

SEQ ID NO: 3 - protein, 7 aa, Synechocystis PCC6803
INCDFLL

SEQ ID NO: 4 - protein, 8 aa, Synechocystis PCC6803
YSTCYFIM